(19) United States
(12) Patent Application Publication
(10) Pub. No.: US 2023/0081468 A1
(43) Pub. Date: Mar. 16, 2023

(54) SYSTEM FOR EVALUATING A BATCH OF MEDICAMENT DELIVERY DEVICES, AND METHOD

(71) Applicant: SHL MEDICAL AG, ZUG (CH)

(72) Inventor: Antonio Farieta, Baar (CH)

(21) Appl. No.: 17/798,642

(22) PCT Filed: Mar. 1, 2021
(86) PCT No.: PCT/EP2021/054967

§ 371 Date: Aug. 10, 2022

(30) Foreign Application Priority Data
Mar. 11, 2020 (EP) 20162291.7

(51) Int. Cl. A61M 5/20; A61M 5/32

(52) U.S. Cl. CPC A61M 5/2033; A61M 5/3202

(57) ABSTRACT

A system for evaluating if a batch of medicament delivery devices should be recalled, wherein the medicament delivery devices are each configured to establish a connection to a remote computer over a communication network when the medicament delivery device is activated; wherein each medicament delivery device is configured to transfer batch data, sensor data and time data to the remote computer; wherein the remote computer is configured to create a batch data set containing the batch data, the sensor data and the time data for the batch; and wherein the remote computer is configured to evaluate the batch data set and the regular reference data stored in the remote memory based on machine learning and to determine, based on the evaluation, if a batch of medicament delivery devices should be recalled.

### CROSS REFERENCE TO RELATED APPLICATIONS

The present application is a U.S. National Phase Application pursuant to 35 U.S.C. § 371 of International Application No. PCT/EP2021/054967 filed Mar. 1, 2021, which claims priority to European Patent Application No. 20162291.7 filed Mar. 11, 2020. The entire disclosure contents of these applications are herewith incorporated by reference into the present application.

### TECHNICAL FIELD

The present disclosure generally relates to medicament delivery devices. In particular, a system for evaluating if a batch of medicament delivery devices should be recalled, and a method of evaluating if a batch of medicament delivery devices should be recalled, are provided.

### BACKGROUND

A wide range of medicament delivery devices for self-administration of medicaments are known. Medicament delivery devices, such as pen injectors, auto-injectors, on-body devices, pumps, and inhalers, typically comprise a housing in which a medicament container containing a medicament is to be arranged. Upon activation of the medicament delivery device, the medicament is expelled through a medicament delivery member, as for example a needle, a cannula or a nozzle.

Some medicament delivery devices are nowadays being provided with functionality that will enable the monitoring and recording of different status changes of the devices when used by the users themselves. The monitoring and recording features will then provide healthcare staff with information on how a user is handling the administration of the medicaments prescribed. Also, users themselves may benefit from the monitoring features, providing a help of remembering and an aid of alerting when a medicament delivery occurrence is scheduled.

For patient safety, it is important that a medicament delivery device is reliable and works as intended. However, such medicament delivery devices are exposed to high temperatures, humidity, long shelf life, shocks during transportation, etc., which may affect the functioning of the devices.

WO 2015187799 A1 discloses systems and methods for processing sensor data collected by a drug delivery device with an external computing device.

### SUMMARY

In the present disclosure, when the term “distal” is used, this refers to the direction pointing away from the dose delivery site. When the term “distal part/end” is used, this refers to the part/end of the medicament delivery device, or the parts/ends of the members thereof, which under use of the medicament delivery device is/are located furthest away from the dose delivery site. Correspondingly, when the term “proximal” is used, this refers to the direction pointing to the dose delivery site. When the term “proximal part/end” is used, this refers to the part/end of the medicament delivery device, or the parts/ends of the members thereof, which under use of the medicament delivery device is/are located closest to the dose delivery site.

One object of the present disclosure is to provide a system for evaluating if a batch of medicament delivery devices should be recalled, which system improves condition monitoring of the medicament delivery devices, e.g. along a supply chain and/or during use.

A further object of the present disclosure is to provide a system for evaluating if a batch of medicament delivery devices should be recalled, which system improves reliability and/or health safety of a user of a medicament delivery device, especially for emergency devices like auto-injectors having a medicament that is critical in some life care areas such as e.g. in allergic shocks, overdoses of certain medicaments, etc.

A further object of the present disclosure is to provide a system for evaluating if a batch of medicament delivery devices should be recalled, which system has a simple design and/or functionality.

A still further object of the present disclosure is to provide a system for evaluating if a batch of medicament delivery devices should be recalled, which system has a reliable design and/or functionality.

A still further object of the present disclosure is to provide a system for evaluating if a batch of medicament delivery devices should be recalled, which system solves several or all of the foregoing objects in combination.

A still further object of the present disclosure is to provide a method of evaluating if a batch of medicament delivery devices should be recalled, which method solves one, several or all of the foregoing objects.

According to one aspect, there is provided a system for evaluating if a batch of medicament delivery devices should be recalled. The system comprises a batch of medicament delivery devices for self-administration of a medicament. Each medicament delivery device comprises a plurality of device components for delivering a medicament; a device processor; a device memory comprising batch data related to the batch; a device transceiver; and a sensor. Each medicament delivery device is configured to use the sensor to generate sensor data related to an actual value for a parameter from an interaction between some or all of the device components during use of the medicament delivery device, to generate time data related to a time point of an activation of the medicament delivery device, and to store the sensor data and time data in the device memory. Further, each medicament delivery device may comprise an electric power source, a switch, and/or electro powered components. The switch may be configured to switch on the electric power source by the actuation of a device component i.e. by manually removing a cap from the device or by manually actuating a switch element.

The system further comprises a remote computer. The remote computer comprises a remote processor; a remote transceiver; and a remote memory containing regular reference data related to a regular reference value for a parameter from a regular interaction between some or all of the device components of a comparable medicament delivery device. Each medicament delivery device is configured to establish a connection to the remote computer over a communication network when the medicament delivery device is activated. Each medicament delivery device is configured to transfer the batch data, the sensor data and the time data to the remote computer via the connection. The remote computer is configured to create a batch data set containing the batch data, the sensor data and the time data for the batch, and to store the batch data set in the remote memory. The remote computer is configured to evaluate the batch data set and the regular reference data stored in the remote memory based on machine learning and to determine, based on the evaluation, if a batch of medicament delivery devices should be recalled.

Each medicament delivery device is thus configured to collect sensor data, related to the performance of the device when the device is activated and/or in use, and to send this sensor data to the remote computer for analysis. By analyzing the sensor data, various malfunctions and/or performance reductions of the batch of medicament delivery devices can be detected and/or predicted. The sensor data is thus representative of at least one condition of the medicament delivery device. For example, it may be concluded that a batch of medicament delivery devices should be recalled if one of the actual parameter values associated with a device component is unacceptable. Whether or not such actual parameter value is unacceptable is determined by means of the machine learning. In this way, health safety of a user, such as a patient, of the medicament delivery device is improved.

The batch data, the sensor data and the time data may be sent from the medicament delivery device by means of the device transceiver. The batch data, the sensor data and the time data may be received by the remote computer by means of the remote transceiver.

The batch data may for example contain information regarding batch number, type of the medicament delivery device, type of any of the device components, type of medicament, medicament volume, medicament filling date and/or final assembly date (e.g. when a medicament container is installed in the medicament delivery device). A batch of medicament delivery devices may be a plurality of medicament delivery devices that are produced at the same site or sites, and/or at the same time (e.g. at the same day).

The medicament delivery devices of the batch may be of any type for self-administration of a medicament. Examples of such medicament delivery devices include pen injectors, auto-injectors and inhalers.

The batch data set collected from each medicament delivery device is subjected to machine learning analysis involving the application of one or more algorithms to detect patterns within the batch data set. The one or more algorithms may comprise one or more mathematical models based on the regular reference data as sample data. Thus, the regular reference data may be used as “training data” for the machine learning. The machine learning analysis may be performed by a big data system, e.g. configured into a server or cloud.

The regular reference data may be provided from comparable medicament delivery devices at user locations and/or at a lab. A comparable medicament delivery device may be a medicament delivery device of the same design but from a different batch. Alternatively, or in addition, a comparable medicament delivery device may be a medicament delivery device of a slightly different design.

The machine learning may be configured to use the regular reference data from a plurality of comparable medicament delivery devices as data to be compared with the sensor data. The machine learning algorithm may thus be improved or “trained” over time as more and more regular reference data is collected. If it is concluded, based on the evaluation, that the sensor data is satisfactory, the sensor data from the batch may be added to the regular reference data in the remote memory.

Each medicament delivery device may comprise a global positioning system (GPS) configured to determine a geographical position of the medicament delivery device when the medicament delivery device is activated. In this case, each medicament delivery device may be configured to store the geographical position in the device memory. Also geographical data related to the geographical position may then be transferred from the medicament delivery device to the remote computer via the connection. In this case, the remote computer may be configured to create a batch data set also containing the geographical data. The geographical data may improve the machine learning evaluation. In addition, the geographical data may be useful for finding a potential point along a supply chain causing performance deteriorations of the medicament delivery devices.

The remote memory may contain irregular reference data related to an irregular reference value for a parameter from an irregular interaction between some or all of device components of a comparable medicament delivery device. In this case, the remote computer may be configured to evaluate the sensor data by comparison with the irregular reference data. For example, if it is concluded, based on the evaluation, that the sensor data is not satisfactory, the sensor data from the batch may be stored in the remote memory as irregular reference data. The machine learning algorithm may thus be improved or “trained” over time also based on collected irregular reference data.

The medicament delivery device comprises the following device components: a housing, a cap, a delivery member, a medicament container, a container holder, an activation element, a driven element, a drive element, a feedback element, a resilient element and a holding and release element. The driven element may be a plunger rod and the drive element may be an energy accumulating element e.g. a resilient element or may be an electrically powered motor. The delivery member may be a needle which is attached or attachable to the medicament container or to the container holder. The activation element may be a button or an actuator sleeve such as a delivery member cover, or a combination thereof. The medicament container may comprise a stopper on which a driven element e.g. the plunger rod is configured to exert a force provided by a drive element e.g. the energy accumulating element. The resilient element may be configured to interact with the activation element.

The sensor may be at least one of a group of sensors which comprises a force sensor, an optical sensor, an acoustic sensor, a position sensor, a pressure

At least one parameter may be indicative of a friction and/or a pressure force from the interaction between at least two device components.

At least one parameter may be indicative of a click sound and/or another specific sound from the interaction between at least two device components.

At least one parameter may be indicative of a displacement and/or a time of displacement of one device component in relation to another device component.

At least one parameter may be indicative of a friction force between the activation element and the holding and release element. In order to measure friction force, the sensor of the medicament delivery device may be a friction sensor, such as a tribosensor functioning based on the triboelectric effect.

Another parameter may be indicative of a friction force between the holding and release element and the driven element i.e. the plunger rod.

A further parameter may be indicative of a force between the activation element and the resilient element, i.e. a pressure force between the activation element and the resilient element In order to measure force, the sensor of the medicament delivery device may be a force sensor.

Yet another parameter may be indicative of a pressure force between the drive member i.e. the energy accumulating element and the driven member i.e. the plunger rod.

A further parameter may be indicative of a click sound from an interaction between the signal generating element and the housing. In order to measure sound, the sensor of the medicament delivery device may be an acoustic sensor.

Another parameter may be indicative of a click sound from an interaction between the driven element i.e. the plunger rod, and the stopper, when the device is activated.

A further parameter may be indicative of a movement of the driven element i.e. the plunger rod, in relation to the housing. In order to measure movement, the sensor of the medicament delivery device may be a position sensor.

Another parameter may be indicative of a movement of the stopper in relation to the medicament container. It may be measured the displacement, and/or the time to achieve certain displacement.

Yet another aspect is that the medicament delivery devices may be pen injectors, auto-injectors, on-body devices, pumps, or inhalers, wherein the interaction between the device components of the medicament delivery devices are mechanical or electromechanical.

According to a further aspect, there is provided a method of evaluating if a batch of medicament delivery devices should be recalled. The method comprises providing a batch of medicament delivery devices for self-administration of a medicament, where each medicament delivery device comprises a plurality of device components for delivering a medicament; a device processor; a device memory comprising batch data related to the batch; a device transceiver; and a sensor. The method comprises, for each medicament delivery device, using the sensor to generate sensor data related to an actual value for a parameter from an interaction between some or all of the device components during use of the medicament delivery device, generating time data related to a time point of an activation of the medicament delivery device, and storing the sensor data and time data in the device memory. The method further comprises providing a remote computer comprising a remote processor; a remote transceiver; and a remote memory containing regular reference data related to a regular reference value for a parameter from a regular interaction between some or all of the device components of a comparable medicament delivery device. The method further comprises establishing a connection to the remote computer over a communication network when the medicament delivery device is activated; transferring the batch data, the sensor data and the time data from each medicament delivery device to the remote computer via the connection; creating a batch data set containing the batch data, the sensor data and the time data for the batch, and storing the batch data set in the remote memory; and evaluating the batch data set and the regular reference data stored in the remote memory based on machine learning and determining, based on the evaluation, if a batch of medicament delivery devices should be recalled. The method may be used with any type of medicament delivery device and system for evaluating if a batch of medicament delivery devices should be recalled, according to the present disclosure.

### BRIEF DESCRIPTION OF THE DRAWINGS

Further details, advantages and aspects of the present disclosure will become apparent from the following embodiments taken in conjunction with the drawings, wherein:

FIG. 1 schematically represents a system comprising a batch of medicament delivery devices and a remote computer;

FIG. 2 schematically represents one example of a medicament delivery device;

FIG. 3 schematically represents a further example of a medicament delivery device; and

FIG. 4: schematically represents a further example of a medicament delivery device.

### DETAILED DESCRIPTION

In the following, a system for evaluating if a batch of medicament delivery devices should be recalled, and a method of evaluating if a batch of medicament delivery devices should be recalled, will be described. The same or similar reference numerals will be used to denote the same or similar structural features.

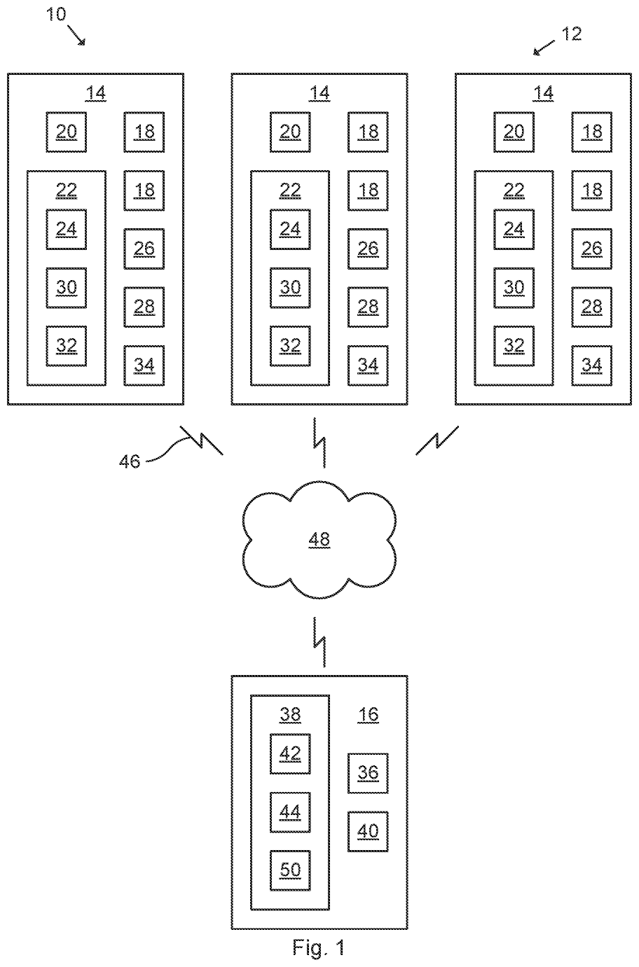

FIG. 1 schematically represents a system 10. The system 10 comprises a batch 12 of medicament delivery devices 14. The system 10 further comprises a remote computer 16. The system 10 is configured to evaluate if the batch 12 of medicament delivery devices 14 should be recalled.

Each medicament delivery device 14 is for self-administration of a medicament. Moreover, each medicament delivery device 14 may be for single or multiple use.

Each medicament delivery device 14 comprises a plurality of device components 18. The device components 18 perform functions associated with medicament delivery from the medicament delivery device 14 to a user.

Each medicament delivery device 14 further comprises a device processor 20 and a device memory 22. The device memory 22 may have a computer program stored thereon, the computer program comprising program code which, when executed by the device processor 20, causes the device processor 20 to perform, or command performance of, various steps as described herein.

The device memory 22 further comprises batch data 24 stored thereon. The batch data 24 may contain various information associated with the batch 12. Examples of such information are batch number, type of the medicament delivery devices 14 of the batch 12, type of any of the device components 18 of the medicament delivery devices 14 of the batch 12, type of medicament contained in the medicament delivery devices 14, volume of medicament contained in the medicament delivery devices 14, a filling time and/or filling date of medicament in the medicament delivery devices 14, and/or a final assembly date of each medicament delivery device 14 of the batch 12. The medicament delivery devices 14 may be considered to belong to the same batch 12 if produced in the same manner, for example at the same production site or sites, with a common machine configuration, and near in time (e.g. during the same day or shift).

Each medicament delivery device 14 further comprises a device transceiver 26 and one or more sensors 28. The sensors 28 are each configured to generate sensor data 30. The sensor data 30 is representative of an actual value of a parameter associated with a performed function of one or more of the device components 18, e.g. a function to activate the device and/or deliver the medicament.

Each medicament delivery device 14 is further configured to generate time data 32, for example by means of the device processor 20. The time data 32 is associated with one or more time points of activation of the medicament delivery device 14, for example when the medicament delivery device 14 is powered on and/or when the medicament is delivered. As shown in FIG. 1, the batch data 24, the sensor data 30 and the time data 32 are stored in the device memory 22. Each medicament delivery device 14 may further comprise an electric power source (not shown) known in the art, such as a battery, energy harvesting means, etc; and a switch (not shown), for electrically powering various components of the medicament delivery device 14.

In this example, each medicament delivery device 14 further comprises a GPS 34. The GPS 34 is configured to determine a geographical position of the medicament delivery device 14 when activated. Geographical data representative of the geographical position, as provided by the GPS 34, is stored in the device memory 22.

The remote computer 16 comprises a remote processor 36, a remote memory 38 and a remote transceiver 40. The remote memory 38 may have a computer program stored thereon, the computer program comprising program code which, when executed by the remote processor 36, causes the remote processor 36 to perform, or command performance of, various steps as described herein.

The remote memory 38 comprises regular reference data 42 stored thereon. The regular reference data 42 is representative of a regular reference value of a parameter associated with a function of one or more of the device components 18, e.g. a function to activate the device and/or to deliver the medicament. The regular reference data 42 may contain regular sensor data from comparable medicament delivery devices of one or more different batches, i.e. sensor data associated with a satisfactory functioning of such medicament delivery devices.

The remote memory 38 may optionally also contain irregular reference data 44 representative of an irregular reference value of the parameter associated with the function of the one or more of the device components 18. The irregular reference data 44 may be associated with an erroneous functioning of comparable medicament delivery devices.

The regular reference data 42 stored in the remote memory 38 and the sensor data 30, as measured by the sensor 28, are associated with the same parameter of the one or more device components 18. More than one such parameters may however be used in the system 10 and with the method.

Each medicament delivery device 14 is further configured to establish a connection 46 with the remote computer 16. As shown in FIG. 1, the connection 46 is established over a communication network 48.

The user may initially activate the device by switching on the electric power source e.g. by means of manually removing e.g. by removing a cap, or by actuating an element/component of the device (not shown). Each medicament delivery device 14 may be configured to automatically establish the connection 46 when powered on. The user can then initiate a medicament delivery procedure, e.g. by pressing the proximal end of the delivery device onto a delivery site and/or by manually actuating a button whereby the device components for delivery the medicament starts to interact. The sensors 28 register actual values of parameters associated with some or all of the device components 18. Values from the sensor 28 during and/or after the medicament delivery procedure are stored as sensor data 30 in the device memory 22. The device transceiver 26 of each medicament delivery device 14 then transfers the batch data 24, the sensor data 30 and the time data 32 via the connection 46 to the remote transceiver 40 of the remote computer 16.

Although not illustrated in FIG. 1, the connection 46 over the communication network 48 may involve a network that allows data traffic as e.g. GPRS, 3G to 5G, LTE or similar cell networks. The connection 46 may involve the use of one or more mobiles phone or other smart appliance. Thus, the batch data 24, the sensor data 30 and the time data 32 may initially be sent from the medicament delivery device 14 to the mobile phone, e.g. via Bluetooth Low Energy (BLE). The batch data 24, the sensor data 30 and the time data 32 may then be sent from the mobile phone to the remote computer 16, e.g. via a cellular network. The mobile phone or other smart appliance may thus form part of the communication network 48. The remote computer 16 may comprise a server or a cloud computer.

The remote computer 16 creates a batch data set 50 containing the batch data 24, the sensor data 30 and the time data 32. In this example, the batch data set 50 also contains the geographical data provided by the GPS 34. As shown in FIG. 1, the batch data set 50 is stored in the remote memory 38.

The remote computer 16 then evaluates the batch data set 50 in view of the regular reference data 42, and optionally also in view of the irregular reference data 44. To this end, the remote computer 16 uses machine learning. The machine learning employs algorithms containing mathematical models based on the regular reference data 42 as training data. In this way, various patterns, such as malfunctions, in the batch data set 50 can be detected and/or predicted. Based on this machine learning evaluation, the remote computer 16 determines whether or not the batch data set 50 is indicative of a satisfactory performance of the medicament delivery devices 14 of the batch 12. The remote computer 16 may create a report, graphs, trend charts and/or generate alerts to the users, manufacturer(s), supply chain, and/or care takers. If not functioning as intended, the remote computer 16 may issue a message, e.g. to the users, manufacturer(s), supply chain, and/or care takers, indicating that the batch 12 should be recalled.

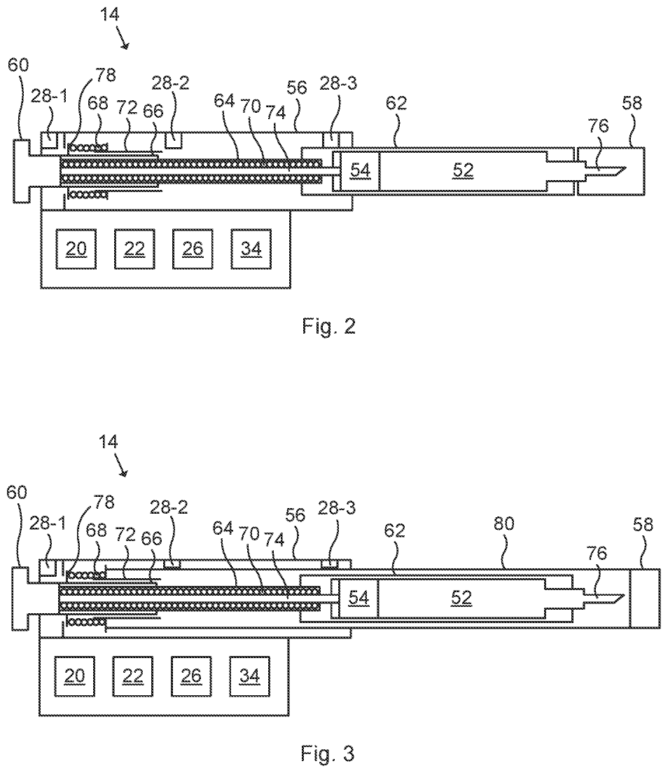

FIG. 2 schematically represents one example of a medicament delivery device 14 in accordance with FIG. 1. The medicament delivery device 14 in FIG. 2 is a pen injector.

The medicament delivery device 14 comprises a medicament container 52 containing medicament. The medicament container 52 comprises a stopper 54. The medicament delivery device 14 further comprises a housing 56, a cap 58 at a proximal end, and an activation element, here exemplified as a button 60, at a distal end. The medicament delivery device 14 further comprises a container holder 62, a plunger rod 64 and a holding and release element 66. The medicament delivery device 14 further comprises two resilient elements, here exemplified as a locking spring 68 and a plunger spring 70. The medicament delivery device 14 of this example further comprises an actuator sleeve 72, a guide rod 74, a needle 76 and a signal generating element 78. The medicament delivery device 14 may also comprise a dose setting element. Each of the medicament container 52, the stopper 54, the housing 56, the cap 58, the button 60, the container holder 62, the plunger rod 64, the holding and release element 66, the locking spring 68, the plunger spring 70, the actuator sleeve 72, the guide rod 74, the needle 76 and the signal generating element 78 may thus constitute a device component 18 according to the present disclosure and as illustrated in FIG. 1. An example of such a medicament delivery device can be found in WO2013058698 and the references cited therein.

As shown in FIG. 2, the medicament delivery device 14 further comprises three sensors 28-1, 28-2 and 28-3. In this example, the sensor 28-1 is an acoustic sensor configured to measure a sound between the signal generating element 78 and the housing 56, the sensor 28-2 is a friction sensor configured to measure a friction force between the holding and release element 66 and the plunger rod 64, and the sensor 28-3 is an acoustic sensor configured to measure a sound between the plunger rod 64 and the stopper 54.

The functioning of the medicament delivery device 14 in FIG. 2 for delivering medicament may be as follows. The user screws the container holder 62 into the housing 56 until the actuator sleeve 72 is pushed distally against the compression of the locking spring 68. The holding and release element 66 is thereby partly exposed from the actuator sleeve 72 inside the housing 56.

The user then removes the cap 58 to expose the needle 76 and pierces the needle 76 into an injection site. When the user pushes the button 60, the holding and release element 66 is pushed proximally further relative the actuator sleeve 72. This causes arms of the holding and release element 66 to release the plunger rod 64. In FIG. 2, a combined manipulation of the container holder 62 and the button 60 is used for activating medicament delivery.

During movement of the plunger rod 64 relative to the holding and release element 66, the friction sensor 28-2 measures the friction and generates corresponding sensor data 30. The plunger rod 64 moves proximally into contact with the stopper 54 and starts to push the stopper 54 to expel the medicament through the needle 76. The acoustic sensor 28-3 measures the sound from the collision between the plunger rod 64 and the stopper 54 and generates corresponding sensor data 30.

When the medicament container 52 is emptied, the plunger spring 70 pushes the holding and release element 66 distally such that the signal generating element 78 collides with the housing 56 to generate a click sound. The acoustic sensor 28-1 measures this sound and generates corresponding sensor data 30.

FIG. 3 schematically represents a further example of a medicament delivery device 14. With reference to FIG. 3, mainly differences with respect to FIG. 2 will be described. The medicament delivery device 14 in FIG. 3 is an auto-injector comprising activation elements exemplified as a button 60 and a needle cover 80. Also the needle cover 80 is a device component 18 according to the present disclosure. In FIG. 3, a combined manipulation of the needle cover 80 and the button 60 is used for activating medicament delivery. The plunger rod 64 can be released by distal movement of the needle cover 80 relative to the housing 56 in combination with pushing the button 60. The needle cover 80 is moved distally relative to the housing 56 by pushing the medicament delivery device 14 against a dose delivery site. An example of such a medicament delivery device can be found in WO02047746 and the references cited therein.

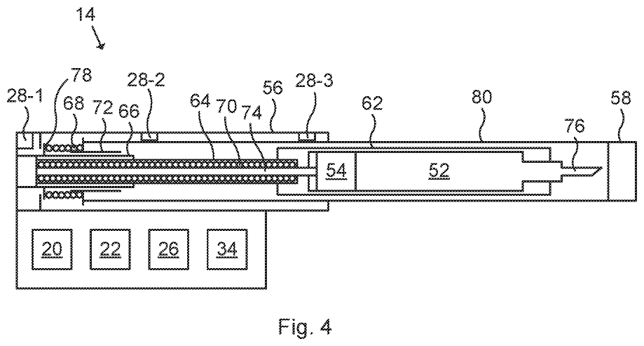

FIG. 4 schematically represents a further example of a medicament delivery device 14. With reference to FIG. 4, mainly differences with respect to FIGS. 2 and 3 will be described. The medicament delivery device 14 in FIG. 4 is an auto-injector. The medicament delivery device 14 does not comprise any button 60. Instead, the needle cover 80 is used as an activation member alone. The medicament delivery device 14 in FIGS. 2-4 are comparable according to the present disclosure. Example of such medicament delivery devices can be found in WO2011123024, WO2013048310 and the references cited therein.

It is also feasible that at least one of the following components such as the electric power source, the device processor 20, the device memory 22, the device transceiver 26 the sensor 28 are arranged in an add-on housing which is fixed or removable attached to medicament delivery device housing 56.

While the present disclosure has been described with reference to exemplary embodiments, it will be appreciated that the present disclosure is not limited to what has been described above. For example, it will be appreciated that the dimensions of the parts may be varied as needed. Accordingly, it is intended that the present disclosure may be limited only by the scope of the claims appended hereto.

1-15. (canceled)

16. A system for evaluating if a batch of medicament delivery devices should be recalled, the system comprising:
a batch of medicament delivery devices for self-administration of a medicament, wherein each medicament delivery device comprises:
a plurality of device components for delivering a medicament;
a device processor;
a device memory comprising batch data related to the batch;
a device transceiver; and
a sensor;
wherein each medicament delivery device is configured to use the sensor to generate sensor data related to an actual value for a parameter from an interaction between some or all of the device components during use of the medicament delivery device, to generate time data related to a time point of an activation of the medicament delivery device, and to store the sensor data and time data in the device memory; and
a remote computer comprising:
a remote processor;
a remote transceiver; and
a remote memory containing regular reference data related to a regular reference value for a parameter from a regular interaction between some or all of the device components of a comparable medicament delivery device;
wherein each medicament delivery device is configured to establish a connection to the remote computer over a communication network when the medicament delivery device is activated;
wherein each medicament delivery device is configured to transfer the batch data, the sensor data and the time data to the remote computer via the connection;
wherein the remote computer is configured to create a batch data set containing the batch data, the sensor data and the time data for the batch, and to store the batch data set in the remote memory; and
wherein the remote computer is configured to evaluate the batch data set and the regular reference data stored in the remote memory based on machine learning and to determine, based on the evaluation, if a batch of medicament delivery devices should be recalled.

17. The system according to claim 16, wherein the machine learning is configured to use the regular reference data from a plurality of comparable medicament delivery devices as data to be compared with the sensor data.

18. The system according to claim 16, wherein each medicament delivery device comprises a GPS configured to determine a geographical position of the medicament delivery device when the medicament delivery device is activated, and wherein each medicament delivery device is configured to store the geographical position in the device memory.

19. The system according to claim 16, wherein the remote memory contains irregular reference data related to an irregular reference value for a parameter from an irregular interaction between some or all of device components of a comparable medicament delivery device, and wherein the remote computer is configured to evaluate the sensor data by comparison with the irregular reference data.

20. The system according to claim 16, wherein at least one parameter may be indicative of a friction and/or a pressure force and/or a click sound and/or another specific sound from the interaction between at least two device components and/or a displacement and/or a time of displacement of one device component in relation to another device component.

21. The system according to claim 16, wherein the sensor is at least one of a group of sensors which comprises a force sensor, an optical sensor, an acoustic sensor, a position sensor, a pressure sensor and a friction sensor.

22. The system according to claim 16, wherein the medicament delivery device comprises an activation element and a holding and release element, and wherein the parameter is indicative of a friction force between the activation element and the holding and release element.

23. The system according to claim 16, wherein the medicament delivery device comprises a holding and release element and a driven element, and wherein the parameter is indicative of a friction force between the holding and release element and the driven element.

24. The system according to claim 16, wherein the medicament delivery device comprises an activation element and a resilient element, and wherein the parameter is indicative of a force between the activation element and the resilient element.

25. The system according to claim 16, wherein the medicament delivery device comprises an energy accumulating element and a driven element, and wherein the parameter is indicative of a force between the energy accumulating element and a driven element.

26. The system according to claim 16, wherein the medicament delivery device comprises a signal generating element and a housing, and wherein the parameter is indicative of a click sound from an interaction between the signal generating element and the housing.

27. The system according to claim 16, wherein the medicament delivery device comprises a driven element and a medicament container having a stopper, and wherein the parameter is indicative of a click sound from an interaction between the driven member and the stopper.

28. The system according to claim 16, wherein the medicament delivery device comprises a driven member and a housing, and wherein the parameter is indicative of a movement of the driven member in relation to the housing.

29. The system according claim 16, wherein the medicament delivery device is a pen injector, an auto-injector, an on-body device, a pump, or an inhaler.

30. A method of evaluating if a batch of medicament delivery devices should be recalled, the method comprising:
providing a batch of medicament delivery devices for self-administration of a medicament, wherein each medicament delivery device comprises:
a plurality of device components for delivering a medicament;
a device processor;
a device memory comprising batch data related to the batch;
a device transceiver; and
a sensor;
for each medicament delivery device, using the sensor to generate sensor data related to an actual value for a parameter from an interaction between some or all of the device components during use of the medicament delivery device, generating time data related to a time point of an activation of the medicament delivery device, and storing the sensor data and time data in the device memory;
providing a remote computer comprising:
a remote processor;
a remote transceiver; and
a remote memory containing regular reference data related to a regular reference value for a parameter from a regular interaction between some or all of the device components of a comparable medicament delivery device;
establishing a connection to the remote computer over a communication network when the medicament delivery device is activated;
transferring the batch data, the sensor data and the time data from each medicament delivery device to the remote computer via the connection;
creating a batch data set containing the batch data, the sensor data and the time data for the batch, and storing the batch data set in the remote memory; and
evaluating the batch data set and the regular reference data stored in the remote memory based on machine learning and determining, based on the evaluation, if a batch of medicament delivery devices should be recalled.

31. A system for evaluating if a batch of medicament delivery devices should be recalled, the system comprising:
a batch of medicament delivery devices for self-administration of a medicament, wherein each medicament delivery device comprises:
a plurality of device components for delivering a medicament;
a device processor;
a device memory comprising batch data related to the batch;
a GPS configured to determine a geographical position of the medicament delivery device when the medicament delivery device is activated;
a device transceiver; and
a sensor comprising at least one of a group of sensors which comprises a force sensor, an optical sensor, an acoustic sensor, a position sensor, a pressure sensor and a friction sensor;
wherein each medicament delivery device is configured to use the sensor to generate sensor data related to an actual value for a parameter from an interaction between some or all of the device components during use of the medicament delivery device, to generate time data related to a time point of an activation of the medicament delivery device, and to store the sensor data and time data in the device memory; and
a remote computer comprising:
a remote processor;
a remote transceiver; and
a remote memory containing regular reference data related to a regular reference value for a parameter from a regular interaction between some or all of the device components of a comparable medicament delivery device;
wherein each medicament delivery device is configured to establish a connection to the remote computer over a communication network when the medicament delivery device is activated;
wherein each medicament delivery device is configured to transfer the batch data, the sensor data and the time data to the remote computer via the connection;
wherein the remote computer is configured to create a batch data set containing the batch data, the sensor data and the time data for the batch, and to store the batch data set in the remote memory; and
wherein the remote computer is configured to evaluate the batch data set and the regular reference data stored in the remote memory based on machine learning and to determine, based on the evaluation, if a batch of medicament delivery devices should be recalled.

32. The system according to claim 31, wherein at least one parameter comprises:
a friction force;
a pressure force;
a click sound; or
a sound generated from the interaction between at least two device components or a displacement of one device component in relation to another device component.

33. The system according to claim 32, wherein the medicament delivery device comprises a driven member and a housing, and wherein the parameter further comprises a movement of the driven member in relation to the housing.

34. The system according to claim 31, wherein the medicament delivery device comprises a signal generating element and a housing, and wherein the parameter comprises a click sound from an interaction between the signal generating element and the housing.

35. The system according to claim 31, wherein the medicament delivery device comprises a needle, a cannula or a nozzle configured to expel medicament from the medicament delivery device.
